(19) United States
(12) Patent Application Publication
(10) Pub. No.: US 2006/0216805 A1
(43) Pub. Date: Sep. 28, 2006

(54) Lactic acid, polylactic acid and biodegradable plastic

(76) Inventor: Shoji Ueda, Sakai-shi (JP)

Correspondence Address: JORDAN AND HAMBURG LLP, 122 EAST 42ND STREET, SUITE 4000, NEW YORK, NY, 10168, US

(21) Appl. No.: 11/181,218

(22) Filed: Jul. 13, 2005

(30) Foreign Application Priority Data
Mar. 24, 2005 (JP) 2005-085240

(51) Int. Cl. C12N 11/00; C12N 11/16

(52) U.S. Cl. 435/174

(57) ABSTRACT

The invention relates to a lactic acid as a raw material of a lactic acid type biodegradable plastic. The invention also relates to a polylactic acid produced using the lactic acid as starting material and a biodegradable plastic produced using this polylactic acid as a part or all of starting material. The lactic acid comprises using starch contained in a coconut as a raw material and lactic-fermenting the starch. The polylactic acid is synthesized using the above lactic acid as starting material. The biodegradable plastic is produced using the above polylactic acid as a part or all of starting material.

### BACKGROUND OF THE INVENTION

1. Field of the Invention

The invention relates to a lactic acid used primarily as a raw material of a lactic acid type biodegradable plastic, to a polylactic acid and a biodegradable plastic produced using this polylactic acid as a part or all of a raw material.

2. Description of the Related Art

There is very little doubt that high-molecular materials such as plastics play a very important role in the chemical and technical progress of the 20th century.

High-molecular materials have functional characteristics superior in, for example, electrical insulating characteristics, dielectric characteristics and light-weight characteristics, are also inexpensive and have such excellent molding characteristics that they can be made into various forms such as a plate, tube, fiber and thin film. Therefore, the advent of engineering plastics improved outstandingly in mechanical strength and heat resistance and the development of complex materials have led to the recent trend of the use of polymers as aircraft, cosmic, automobile or mechanical materials.

Such developments of high-molecular materials result in the distribution of plastic products which have been increased at a growth rate considered to be abnormal to a current amount exceeding 5,000,000 tons/year from about 10,000 tons/year produced just after the World War II. There are various plastic products abundantly around our livings, showing that these plastic products are closely related to our life.

However, when such plastic products are dumped, it is difficult to separate each plastic in an unmixed state from waste. It is therefore difficult to recover and reuse these plastic products. Also, these plastic products are stable in the air, water and the like without being decomposed by microorganisms and are scarcely decomposed and dissolved, so that they keep their original shapes for a long period of time and are left almost permanently in the environment. It is therefore undesirable to dispose of these waste plastic products, for example, by burying these products. Moreover, when these plastic products are burned, large heat is produced, which gives damages to the inside of the incinerator, requires a large amount of air, and causes the generation of toxic gas and cokes. It is therefore difficult to burn these plastic products completely and it is therefore difficult to treat these plastic products by incineration.

It is natural that the amount of plastics in waste (refuse) is increased with an increase in the amount of plastic products to be used. In these days when more strict regulations are issued for improving environmental problems and refuse treatment problem, it is an unavoidable object to solve the problem as to the treatment of plastic waste. There is a strong demand for the development of functional materials substituted for these plastic products.

For this, biodegradable plastics which will become extinct in a natural environment attract remarkable attention as an ideal measures solving the problem as to the treatment of plastic waste. Considerable research is still ongoing to develop these biodegradable plastic in big enterprises and chemical product manufacturers.

These biodegradable plastics are roughly classified into three categories, that is, “a microbial production system”, “a natural high-molecular system” and “a chemical synthetic system” by the type of material and production method.

In the case of biodegradable plastics belonging to the microbial production type among these categories, grain starch and the like are supplied to specific microorganisms which accumulate polyesters inside the body by metabolism and when polyesters are accumulated in the body of these microorganisms, these polyesters are taken out as a plastic raw material.

However, the microbial production type biodegradable plastic has the problem that it has inferior moldability and a unique odor though it has very good biodegradability and it is therefore limited in the range of its application.

Biodegradable plastics belonging to the natural high-molecular type are those which are designed to exhibit a certain level of strength by using a natural high polymer such as starch, chitosan or a protein as a major raw material to carry out modification or blending treatment of these natural polymers and have been studied from the most initial stage in the field of researches of degradable plastics.

However, this natural high-molecular type biodegradable plastic has the drawback that it generally tend to absorb moisture and its properties are therefore largely dependent on a variation in production circumstances.

For this, the current trend of studies in the field of biodegradable plastics is towards the researches and developments of chemical synthetic types. Among these synthetic types, lactic acid type biodegradable plastics superior in moldability, high rigidity, transparency and safety to living bodies are most expected and a part of these plastics have been already put to practical use (for example, Monthly ASCII, the November issue (2002) and D & M, Nikkei Mechanical, the August issue (2002)).

The lactic acid type biodegradable plastic is a polymer obtained from a lactic acid (L-lactic acid and D-lactic acid) monomer which is a fermented product obtained by using starch as starting material and by subjecting this starch to a lactic fermentation process. This polymer is produced from lactide which is a dimer of lactic acid in general according to a ring-opening polymerization method or a direct polymerization condensation method.

### SUMMARY OF THE INVENTION

Starch to be used as the raw material of lactic acid type biodegradable plastics is usually produced from grain starch contained in agricultural products such as potatoes, sweet potatoes, corns or sugarcanes. These agricultural products have the problem that each crop of these products is changed by the weather and seasons and it is therefore difficult to supply these products stably all the year round.

Also, it takes a relatively long time to obtain lactic acid by fermenting such grain starch, giving rise to the problem that it is difficult to mass-produce lactic acid.

Moreover, because these agricultural products are foods, the use of such foods as industrial raw materials itself has a fundamental problem in these days when there is a problem concerning the gap of food situation between countries.

In view of this situation, the inventors of the present invention have made earnest studies to solve the above problem and, as a result, developed a lactic acid of the present invention by using starch in a coconut as a raw material and at the same time, a polylactic acid using this lactic acid as a raw material and a biodegradable plastic produced using this polylactic acid as a part or all of a raw material.

Specifically, the inventors of the present invention direct their attentions to the point that a lot of starch is contained in albumen and coconut water contained in a coconut and is lactic-fermented very more easily than grain starch, to find that if such a coconut is used, lactic acid to be used as the raw material of a biodegradable plastic can be obtained in a relatively short time.

Almost all coconuts are currently utilized as follows: for example, in Sri Lanka, only coconut milk and coconut oil are pressed out of the albumen of harvested coconuts and almost all of a squeezed albumen residue and coconut water are dumped. The amount of the dumped waste reaches 40×10⁴ to 50×10⁴ t per day, to find that it is possible to obtain a lot of lactic acid if these squeezed albumen residue and coconut water which are waste material of coconuts are utilized.

The present invention is completed based on the above findings and it is an object of the present invention to provide a lactic acid as a raw material of a lactic acid type biodegradable plastic. Another object of the present invention is to provide a polylactic acid using the above lactic acid as a raw material. A further object of the present invention is to provide a biodegradable plastic produced using the polylactic acid as a part or all of a raw material.

The above object is attained by a lactic acid according to the present invention, the lactic acid being produced by using starch in a coconut as a raw material and by subjecting this coconut to a lactic fermentation process.

### DESCRIPTION OF THE PREFERRED EXAMPLES

The reason why “starch in a coconut” like this is used as the raw material of the lactic acid of the present invention is that as mentioned above, a lot of starch is contained in albumen and coconut water contained in a coconut and lactic-fermented very more easily than grain starch, and if such starch in a coconut is used, lactic acid to be used as the raw material of a biodegradable plastic can be obtained in a relatively short time.

The reason is also that almost all coconuts are currently utilized as follows: for example, in Sri Lanka, only coconut milk and coconut oil are pressed out of the albumen of harvested coconuts and almost all of a squeezed albumen residue and coconut water are dumped and the amount of the dumped waste reaches 4×10⁴ to 50×10⁴ t per day, so that it is possible to obtain a lot of lactic acid if these squeezed albumen residue and coconut water which are coconut waste are utilized.

Accordingly, in the present invention, particularly a starch in squeezed albumen residue and coconut water which are the waste of a coconut is preferably used as a raw material and this starch is subjected to a lactic fermentation process from the viewpoint of utilizing waste products.

As a method of lactic-fermenting starch in a coconut, conventionally known measures, for example, the same measures that are used to lactic-ferment grain starch such as potatoes and corns may be used. Specifically, a method may be exemplified in which a proper amount of lactic bacteria is added to the raw material and the fermentation condition and fermentation temperature of each lactic bacterium, the pH of coconut water and fermentation time are properly controlled to allow starch in a coconut to produce lactic acid by the metabolism of the lactic bacteria.

At this time, various nutrients including starch and an acid, an alkali and the like for adjusting the pH may be properly added and the fermentation may be mixing fermentation or continuous fermentation.

Then, the fermented mixture is subjected to filtration and may be, as desired, subjected to refining processes such as deodorizing and decoloring processes utilizing a sterilizing process, pH regulation, an ion-exchange resin, an activated carbon column, a dialysis membrane and the like.

Here, the term “lactic bacteria” mean Lactobacillaceae Lactobacillus, Actinomycetaceae Bifidobacterium, Sporobacillaceae Sporolactobacillus and Streptococcaceae Pediococcus, Enterococcus faecalis, Streptococcus and Leuconostoc. Examples of the above “Lactobacillaceae Lactobacillus” may include bacteria belonging to the genus Lactobacillaceae Lactobacillus: Lactobacillus acidophilus, Lactobacillus brevis, Lactobacillus bulgaricus, Lactobacillus casei, Lactobacillus delbruckii, Lactobacillus fermenti, Lactobacillus helveticus, Lactobacillus jugurti, Lactobacillus lactis and Lactobacillus plantarum.

Examples of the above “Actinomycetaceae Bifidobacterium” may include bacteria belonging to the genus Actinomycetaceae Bifidobacterium: Bifidobacterium adolescentis, Bifidobacterium bifidum, Bifidobacterium breve, Bifidobacterium infantis, Bifidobacterium lactentis, Bifidobacterium liberorum, Bifidobacterium longum, Bifidobacterium parvulorum, Bifidobacterium pseudolongum and Bifidobacterium thermophilum. Examples of the above “Sporobacillaceae Sporolactobacillus” may include bacteria belonging to the genus Sporobacillaceae Sporolactobacillus: Sporolactobacillus inulinus.

Examples of the above “Streptococcaceae Pediococcus” include bacteria belonging to the genus Streptococcaceae Pediococcus and lactic bacteria belonging to the genus Streptococcaceae Enterococcus faecalis: Pediococcus acidilactis, Pediococcus cerevisiae, Pediococcus halophilus and Pediococcus pentosaceus. Examples of the above “Streptococcaceae Streptococcus” include bacteria belonging to the genus Streptococcaceae Streptococcus: Streptococcus cremoris, Streptococcus diacetilactis, Streptococcus faecalis, Streptococcus faecium, Streptococcus lactis, Streptococcus lactis sub-sp. diacetylactis, Streptococcus thermbphilus and Streptococcus uberis.

Examples of the above “Streptococcaceae Leuconostoc” may include bacteria belonging to the genus Streptococcaceae Leuconostoc: Leuconostoc citrovorum, Leuconostoc cremoris, Leuconostoc dextranicum and Leuconostoc mesenteroides.

Other than the above bacteria, congeners of the above lactic bacteria may also be used in the present invention and also desired mating species of these bacteria may also be used.

The lactic acid in the present invention can be obtained by extracting lactic acid after starch in a coconut is lactic-fermented sufficiently.

As lactic acid, optical isomers D-lactic acid and L-lactic acid are present. In the present invention, a mixture of D-lactic acid and L-lactic acid may be extracted and the extracting operation may be further forwarded to isolate D-lactic acid from L-lactic acid.

The polylactic acid of the present invention is characterized in that it is produced using the above lactic acid of the present invention as a raw material. Specifically, the polylactic acid is a polymer produced using the above lactic acid of the present invention as a monomer.

The polylactic acid of the present invention is usually produced from lactide which is a dimer of lactic acid by a ring-opening polymerization method and a direct polymerization condensation method.

The biodegradable plastic of the present invention is characterized in that it is produced using the above polylactic acid of the present invention as a part or all of a raw material. Specifically, the biodegradable plastic may be produced using the polylactic acid of the present invention singly or as a blended product with other aliphatic polyesters or reins.

Here, examples of the other aliphatic polyesters and resins may include, though not limited to, polyhydroxyalkanoates such as 3-hydroxybutyrate, 3-hydroxyvalerate, 3-hydroxycaproate, 3-hydroxyheptanoate, 3-hydroxyoctanoate, 3-hydroxynanoate, 3-hydroxydecanoate, γ-butyrolactone, δ-valerolactone and ε-caprolactone or copolymers of these alkanoates.

Also, the biodegradable plastic of the present invention may be used as a laminate with other resins as desired. For example, a gas-barrier resin such as an ethylene/vinyl alcohol copolymer and methaxylylene adipamide (MXD6) is used as a laminate with the biodegradable plastic in applications which need barrier characteristics against oxygen and a moisture-barrier resin such as a cyclic olefin polymer is used as a laminate with the biodegradable plastic in applications which need barrier characteristics against moisture. Moreover, it is possible to provide a coating layer made of a metal oxide to improve gas barrier characteristics.

In the meantime, the biodegradable plastic in the present invention may be utilized as, for example, materials in agriculture, forestry and fishery fields (e.g., films, vegetable cultivation pots, fishing lines and fishing nets), materials in civil works (e.g., moisture-retentive sheets, plant nets and sand bags) and materials in package and container fields (materials that are recycled with difficulty because soils and foods are usually attached thereto) and also applied to, for example, casings of electric products or electronic devices and parts of electronic devices.

However, when a biodegradable plastic according to the present invention is applied to, for example, and casings of electric products and electronic devices, it is sometimes unsatisfactory in the point of long-term reliability in an actual circumstance where it is used in various temperature and humidity conditions.

This is because active hydrogen in a functional group having active hydrogen such as a carboxyl group and a hydroxyl group in the polylactic acid hydrolyzes the primary chain catalytically, causing a deterioration in properties such as heat resistance and impact resistance.

Therefore, in order to maintain the properties of the biodegradable plastic of the present invention, it is preferable to add a compound (hereinafter abbreviated as a hydrolysis inhibitor), such as a carbodiimide compound, isocyanate compound or oxazoline type compound, which is reactive with a carboxyl group, a hydroxyl group in the polylactic acid or an amino group or/and hydrogen of an amide bond in the degradable polymer contained as a copolymer or a mixture.

Among these compounds, a carbodiimide compound is preferable because it can be easily melt-kneaded with a polyester and the hydrolytic property can be controlled by adding this compound in a small amount. These hydrolysis inhibitors may be used either singly or in combinations of two or more.

Examples of the above carbodiimide compound may include dicyclohexylcarbodiimide, diisopropylcarbodiimide, dimethylcarbodiimide and diisobutylcarbodiimide. Examples of the above isocyanate compound include 2,4-tolylenediisocyanate, 2,6-tolylenediisocyanate, m-phenylenediisocyanate, p-phenylenediisocyanate and 4,4′-diphenylmethanediisocyanate. Examples of the above oxazoline type compound may include 2,2′-o-phenylenebis(2-oxazoline), 2,2′-m-phenylenebis(2-oxazoline), 2,2′-p-phenylenebis(2-oxazoline) and 2,2′-p-phenylenebis(4-methyl-2-oxazoline).

As a usual method of treating the biodegradable plastic of the invention by using the hydrolysis inhibitor, a method in which the hydrolysis inhibitor is added before, when or after a polyester is melted and then melted to mix it with the degradable plastic is used.

Here, as to the long-term reliability and biodegradation speed (after used) of the biodegradable polyester treated by the hydrolysis inhibitor, the delay of these properties can be controlled by the type and amount of the hydrolysis inhibitor to be compounded. The type and amount of the hydrolysis inhibitor to be compounded may be decided corresponding to the mechanical strength required for an intended product. However, the usual amount of the hydrolysis inhibitor to be added is preferably about 0.1 to 5% by weight based on the total amount of the biodegradable plastic.

A reinforcing material may be compounded in the biodegradable plastic of the present invention to reinforce the properties and mechanical strength such as impact resistance.

Examples of the reinforcing material may include, though not limited to, inorganic or organic fillers. Examples of the above inorganic type filler may include carbon, silicon dioxide and silicates such as zeolite, whereas examples of the above organic type filler may include natural rubbers and biodegradable elastomer materials.

The above reinforcing materials may be used either singly or by mixing two or more. The amount of the reinforcing material to be added is preferably about 5 to 30% by weight based on the biodegradable plastic according to the present invention in general though it may be optionally designed according to the type and intended strength.

In the meantime, in order to use the biodegradable plastic of the present invention as casings of electric products, it is necessary to fulfill the flame retardation standards prescribed in Japanese Industrial Standard (JIS) and UL (Under-writer Laboratory) Standard.

For this, in order to raise the safety of the biodegradable plastic of the present invention when the plastic is used, it is preferable to add a flame retardant additive to thereby impart flame retardation.

Examples of the flame retardant additive may include, though not limited to, hydroxide type compounds, ammonium phosphate type compounds and silica type compounds.

The hydroxide type compound absorbs the heat generated when the resin is burned and is decomposed and produces water at the same time to develop flame retardation by these heat absorbing action and generation of water. Also, the ammonium phosphate type compound is decomposed when the resin is burned to produce polymethaphosphoric acid. As a result of the dehydration effect, a new carbon film which prevents the permeation of oxygen is formed, producing a flame retarding effect. Moreover, the silica type compound imparts flame retardation to the resin by the effect of the inorganic filler on the resin.

Examples of the above hydroxide type compound include aluminum hydroxide, magnesium hydroxide and calcium hydroxide.

Also, examples of the ammonium phosphate type compound may include ammonium phosphate and ammonium polyphosphate.

Moreover, examples of the above silica type compound may include silicon dioxide, low-melting point glass and organosiloxane.

The amount of these flame retardant additives to be added may be decided arbitrarily, though not limited, within a range where the mechanical strength of the biodegradable plastic of the present invention can be secured. Specifically, the amount of the flame retardant additive is preferably about 5 to 50% by weight based on the biodegradable plastic in the case where the flame retardant additive is a hydroxide type compound, about 2 to 40% by weight based on the biodegradable plastic in the case where the flame retardant additive is an ammonium phosphate type compound and about 5 to 30% by weight based on the biodegradable plastic in the case where the flame retardant additive is a silica type compound.

In addition, other known additives may be contained in the biodegradable plastic of the present invention. Examples of these additives may include an antioxidant, heat stabilizer, ultraviolet absorber, lubricant, waxes, colorant, crystallization promoter and degradable organic materials such as starch. These materials may be used either singly or in combinations of two or more. These additives are preferably harmless compounds taking environmental safeguard into account.

The biodegradable plastic of the present invention may be used in various applications, for example, molded articles such as materials in agriculture, forestry and fishery fields (e.g., sheets, films, vegetable cultivation pots, fishing lines and fishing nets), materials in civil works (e.g., moisture-retentive sheets, plant nets and sand bags) and materials in package and container fields (materials that are recycled with difficulty because soils and foods are usually attached thereto) and also applied to, for example, other applications such as casings of electric products and electronic devices such as radios, mikes, TVs, keyboards, portable type music regenerators and personal computers.

Examples of the method of producing the above molded articles include film molding, extrusion molding and injection molding. To state in more detail, the above extrusion molding may be carried out using a known extrusion molding machine, for example, a single shaft extruder, multi-shaft extruder or tandem extruder according to a usual method. Also, the above injection molding may be carried out using a known injection molding machine such as an inline screw-type injection molding machine, multilayer injection molding machine or two-head type injection molding machine.

The present invention relates to a novel lactic acid which has the above structure and is obtained by fermenting starch in a coconut, polylactic acid, and a biodegradable plastic produced using this polylactic acid as a part or all of a raw material.

Specifically, the lactic acid of the present invention is produced using starch in a coconut as a raw material. A lot of starch is contained in albumen and coconut water contained in a coconut and is lactic-fermented more easily than grain starch, and lactic acid to be used as the raw material of a biodegradable plastic can be obtained in a relatively short time.

Also, almost all coconuts are currently utilized as follows: only coconut milk and coconut oil are pressed out of the albumen of harvested coconuts and almost all of a squeezed albumen residue and coconut water are dumped and the amount of the dumped waste reaches 40×10⁴ to 50×10⁴ t per day. According to the present invention, it is possible to utilize these squeezed albumen residue and coconut water which are waste material of such coconuts and also to obtain a lot of lactic acid.

### EXAMPLES

The present invention will been explained in detail by way of examples, which are not intended to be limiting of the present invention.

Lactic bacterium was added to coconut water and an albumen squeezed residue and the mixture was allowed to stand for a fixed period of time to proceed lactic fermentation, thereby obtaining lactic acid.

The obtained lactic acid was a mixture of a D-isomer and a L-isomer. First, this lactic acid was pre-polymerized to thereby obtain lactide (LL-lactide, LD-lactide and DD-lactide) including three types of stereoisomers which were intermediate materials.

Next, the above lactide was subjected to vacuum distillation by a known method to run ring-opening polymerization, thereby obtaining a polylactic acid.

The resulting polylactic acid had the advantage that it was a complete recycle type resin which was decomposed into water and carbon dioxide gas by microorganisms existing in the natural word. Also, the polylactic acid had a glass transition point (Tg) of about 60° C. close to the Tg of polyethylene terephthalate (biodegradable plastic).

What is claimed is:

1. A lactic acid comprising using starch contained in a coconut as a raw material and lactic-fermenting the starch.

2. A polylactic acid synthesized using the lactic acid as claimed in claim 1 as a raw material.

3. A biodegradable plastic produced using the polylactic acid as claimed in claim 2 as a part or all of a raw material.
